(19) United States
(12) Patent Application Publication
(10) Pub. No.: US 2006/0096039 A9
(43) Pub. Date: May 11, 2006

(54) Composition comprising at least one conductive polymer and at least one oxidizing agent, and process for treating keratin fibers employing it

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,337

(22) Filed: Jul. 16, 2004

(30) Foreign Application Priority Data
Jul. 16, 2003 (FR) 03 08672

(51) Int. Cl. A61K 7/13

(52) U.S. Cl. 8/405

(57) ABSTRACT

A composition comprising, in a cosmetically acceptable medium, (a) at least one oxidizing agent and (b) at least one conductive polymer, for example, comprising at least one repeating unit chosen from aniline, pyrrole, thiophene, bisthiophene, furan, para-phenylene sulfide, para-phenylene vinylene, indole, aromatic amide, aromatic hydrazide, aromatic azomethine, and aromatic ester. A process for treating fibers, for example, a bleaching, a permanent-reshaping, and a dyeing process, using the composition. The use of this composition for imparting an optical effect on keratin fibers.

Disclosed herein is a composition comprising, in a cosmetically acceptable medium, at least one conductive polymer and at least one oxidizing agent. Further disclosed herein is a process for treating keratin fibers, for example, a bleaching, a permanent-waving, and a dyeing process, using the abovementioned composition. Also disclosed is the use of such a composition to give keratin fibers at least one optical effect.

The present disclosure relates to the field of treating keratin fibers, for example, human keratin fibers, such as hair. For example, disclosed herein are processes for treating keratin fibers using compositions comprising at least one oxidizing agent.

Keratin fibers may be subjected to various treatments, including treatments involving an oxidizing compound, such as treatments for bleaching, permanently reshaping and dyeing keratin fibers.

With regard to bleaching, this treatment may be performed by oxidizing “melanin” pigment, resulting in the dissolution of and partial or total removal of the pigment from the fiber.

To oxidize a “melanin” pigment, bleaching compositions comprising a peroxygenated reagent and an alkaline agent as an activator for these peroxygenated salts may be used. These compositions may be combined, at the time of use, with an aqueous hydrogen peroxide composition.

With regard to permanent-reshaping processes, and, for example, the fixing step of such processes, the fixing step may be performed after shaping the fibers (curling or smoothing out). This first step, during which the disulfide bridges present in the fibers are opened, usually takes place in the presence of a reducing agent. Before, after or simultaneously with the reduction of these disulfide bridges, the fibers may be shaped in the desired manner (curling the fibers or smoothing out the fibers). Once this first step has been performed, it may be necessary to perform a step during which the disulfide bridges are reformed in order to stabilize the shape obtained.

Moreover, in the field of dyeing, it may very often be required to use an oxidizing agent.

For example, in the field of oxidation dyeing, the compounds used are oxidation dye precursors, i.e., oxidation bases optionally combined with at least one coupler. These compounds may be colorless or weakly colored substances, which, in the presence of an oxidizing agent form, via a process of oxidative condensation, compounds that color the fibers.

In the field of semi-permanent dyeing using direct dyes, which may be coloring or colored compounds, the presence of an oxidizing agent may be required when it is desired to lighten the fibers.

Whatever the desired objective (bleaching, reshaping or dyeing), these processes can be relatively aggressive towards the keratin fiber and, after treatments of this type, degradation of the keratin fibers may be observed over time, which may lead to the fibers becoming somewhat coarse, brittle, and dull.

It is possible to treat fibers with an agent that provides them, for example, with sheen; it being possible for the agent either to be present in the bleaching composition, in the permanent-waving fixing composition, in the oxidizing composition used during dyeing steps, or in a composition applied after these treatments.

For example, to impart sheen on the hair, lubricating hydrophobic substances, such as organic oils, waxes, and silicones may, for example, be used. However, the sheen effect obtained may lack intensity and may give hair an artificial look.

In addition, such compositions, once applied to the hair, may have the drawback of giving the hair a greasy or tacky feel.

Finally, in the case where the bleaching or permanent-reshaping step is followed by dyeing, the presence of these compounds may limit the uptake of the dye into the fibers and consequently give less intense or less fast colorations.

One embodiment relates to a composition comprising at least one oxidizing agent and at least one conductive polymer, which may give treated keratin fibers at least one particular optical effect without at least one some of the drawbacks encountered with prior art compositions.

Moreover, when the at least one conductive polymer absorbs in the range of the visible spectrum, the composition disclosed herein may make it possible to color the fibers without a particular dyeing treatment being necessary subsequent to the fiber shaping treatment.

In the case where the oxidizing composition is used in a dyeing process, the presence of the at least one conductive polymer may make it possible to optimize the content of oxidation dye precursors or of direct dyes.

Also, the keratin fibers may have a soft, pleasant feel after the composition disclosed herein is applied.

Disclosed herein is thus a composition comprising, in a cosmetically acceptable medium:
(a) at least one oxidizing agent and
(b) at least one conductive polymer.

Further disclosed herein is process for treating keratin fibers, comprising applying at least one composition, comprising in a cosmetically acceptable medium:
(a) at least one oxidizing agent and
(b) at least one conductive polymer.

Further disclosed herein is a process for bleaching keratin fibers, for example, human keratin fibers, such as hair, comprising:
(a) mixing, before application to the keratin fibers,
   at least one composition comprising in a cosmetically acceptable medium:
      (i) at least one oxidizing agent and
      (ii) at least one conductive polymer, wherein the at least one composition is free of hydrogen peroxide and
   (ii) at least one aqueous composition comprising hydrogen peroxide, wherein the compositions are optionally mixed in the presence of at least one alkaline agent,
b) applying the mixture to wet or dry keratin fibers;
c) leaving the mixture on the fibers for a time period that is sufficient for the desired bleaching effect to take place;
d) optionally rinsing the fibers;
e) optionally washing the fibers and rinsing the fibers; and
f) drying the fibers or leaving the fibers to dry.

Further disclosed herein is a process for permanently reshaping keratin fibers, for example, human keratin fibers, such as hair, comprising:
a) applying to the fibers at least one reducing composition comprising at least one reducing agent;
b) leaving the at least one reducing composition on the fibers for a time period that is sufficient to shape the fibers;
c) optionally rinsing the fibers;
d) applying to the fibers at least one composition comprising, in a cosmetically acceptable medium,
   (i) at least one oxidizing agent and
   (ii) at least one conductive polymer;
b) optionally rinsing the fibers;
c) optionally washing the fibers and rinsing the fibers; and
d) drying the fibers or leaving the fibers to dry.

Even further disclosed herein is a process for dyeing keratin fibers, for example, human keratin fibers, such as hair, comprising:
a) mixing, before application to the keratin fibers,
   at least one dye composition comprising at least one dye chosen from oxidation dye precursors and direct dyes and
   at least one aqueous composition comprising, in a cosmetically acceptable medium,
      (i) at least one oxidizing agent and
      (ii) at least one conductive polymer, wherein the compositions are mixed in the presence of at least one alkaline agent;
b) applying the mixture to the wet or dry keratin fibers;
c) leaving the mixture on the fibers for a time period that is sufficient for the desired dyeing effect to take place;
d) optionally rinsing the fibers;
e) optionally washing the fibers and rinsing the fibers; and
f) drying the fibers or leaving the fibers to dry.

Further disclosed herein is the use of the composition comprising at least one oxidizing agent and at least one conductive polymer, to impart at least one optical effect on keratin fibers.

In one embodiment, the composition disclosed herein uniformly gives keratin fibers at least one particular optical effect, for example, sheen that may be at least one of substantially more intense, more natural, and more aesthetic than with the means of the prior art.

Moreover, when the at least one conductive polymer, present in the composition disclosed herein, absorbs in the visible spectrum, at least one optical effect, for example, chosen from sheen and color may be obtained simultaneously.

In the text hereinbelow and unless otherwise indicated, the limits of a range of values are understood as forming part of that range.

As used herein, the term “optical effect” means sheen, color, metallic, goniochromatic, and moiré effects.

Moreover, it should be noted that, as used herein, “sheen” corresponds to the light intensity reflected at an angle α when the lock of hair is illuminated under an angle -α. The angle α used to measure this specular reflection, in other words, the sheen, is equal to 20°. This provision of sheen is measured using a glossmeter as described in ISO standard 2813-1994 from AFNOR (August 1994, amended February 1997).

Conductive Polymers

As used herein, the term “conductive polymer” means a molecular structure in which the monomer(s) has (have) high electron delocalization and whose arrangement in the polymer skeleton allows the π orbitals to overlap. This chemical characteristic is reflected by electrical conduction, which may or may not be accompanied by absorption in the UV-visible spectrum, or even in the infrared spectrum.

As used herein, the expression “conductive polymer absorbing in the visible spectrum” means any conductive polymer having a non-zero absorbance in the wavelength ranging from 400 to 800 nm, even if the absorption maxima of the polymer is outside this range.

The at least one conductive polymer used in the composition disclosed herein is chosen from conductive polymers that are soluble or dispersible in the cosmetic medium suitable for use.

The at least one conductive polymer is soluble in the medium when it forms an isotropic clear liquid at 25° C. in a medium chosen from water and mixtures of water and at least one solvent, wherein the solubility is obtained throughout all or part of a concentration ranging from 0.01% to 50% by weight of the at least one conductive polymer.

In one embodiment, the at least one conductive polymer is chosen from conductive polymers that are soluble or dispersible in an aqueous medium, for example, in water.

The at least one conductive polymer is dispersible in the medium comprising water and mixtures of water and at least one solvent if, at 0.01% by weight, at 25° C., it forms a stable suspension of fine, generally spherical particles. The mean size of the particles constituting the dispersion is less than 1 μm, and may, for example, range from 5 to 400 nm and, further, for example, from 10 to 250 nm. These particle sizes are measured by light scattering.

It should be noted that, in some embodiments, the at least one conductive polymer does not require the use of a dispersant.

The at least one conductive polymer may, for example, be in a form that is soluble in the medium of the composition.

Further, the at least one conductive polymer may, for example, have a conductivity ranging from 1×10⁻⁵ to 5×10⁵ siemens/cm, for example, from 1×10⁻³ to 1×10⁵ siemens/cm and, further, for example, from 1×10⁻¹ to 1×10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space d, are applied by simple pressure to the sample to be analysed. A current I is injected via the outer points using the current source, thus creating a variation in potential. The voltage U is measured between the two inner points connected to the voltmeter of the current generator.

In this configuration, the conductivity of the sample expressed in S/cm is given by the following expression:
σ−=(K×I)/(U×e)
wherein:
K is a coefficient depending on the position of the contacts on the surface of the sample. When the points are aligned and equidistant, K is equal to: π/log(2);
I is the value of the injected current, expressed in amperes;
U is the measured voltage value, expressed in volts; and
e is the thickness of the sample, expressed in cm.

This expression can be used only when the thickness of the material is negligible compared with the distance d existing between two points (e/d<0.25). In order to obtain sufficiently small thicknesses and thus to be able to calculate the conductivity of the material, it is recommended to perform the measurement on a non-conductive support (for example a glass slide) coated with the material to be analysed, obtained by evaporation of a dilute solution. In order to improve the homogeneity of the coating to be analysed, it is also recommended to use the deposition technique known as spin coating.

In one embodiment, the at least one conductive polymer present in the composition disclosed herein is chosen from polymers comprising at least one repeating unit of the following formulae:
anilines of formula (I) below: 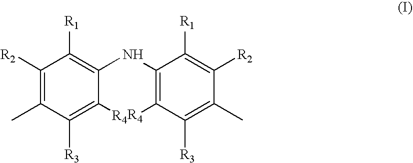
pyrroles of formulae (IIa) and (IIB) below: 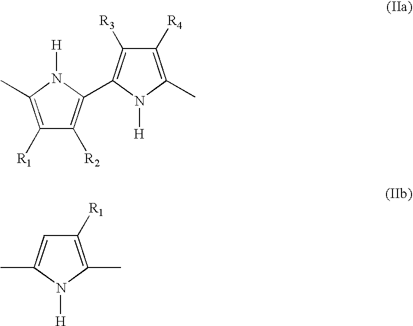
thiophenes and bisthiophenes of formulae (IIIa), (IIIb) and (IIIc) below: 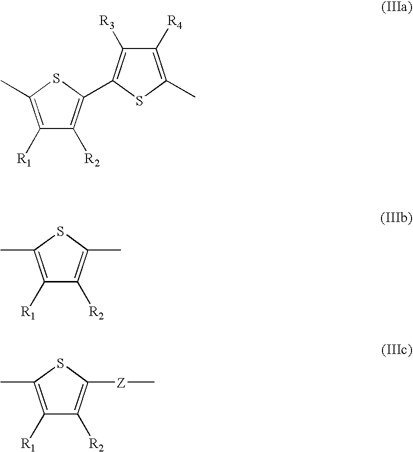
furans of formula (IV) below: 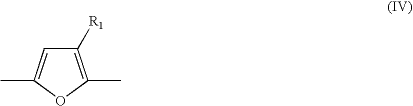
para-phenylene sulfides of formula (V) below: 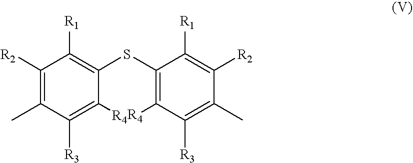
para-phenylenevinylenes of formula (VI) below: 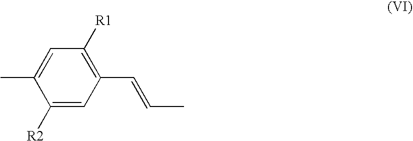
indoles of formula (VII) below: 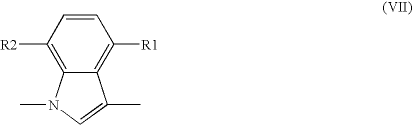
aromatic amides of formulae (VIIIa), (VIIIb), (VIIIc) and (VIIId) below: 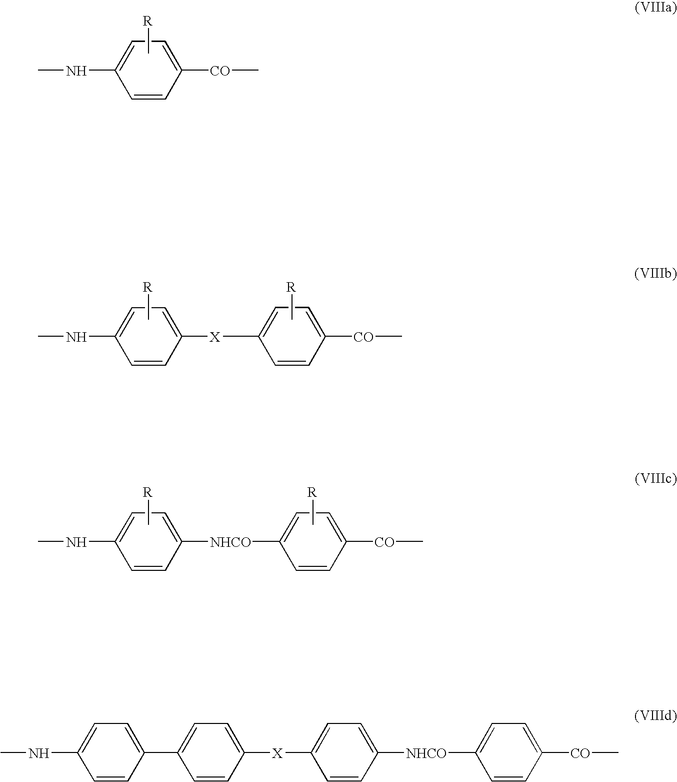
aromatic hydrazides of formulae (IXa), (IXb) and (IXc) below: 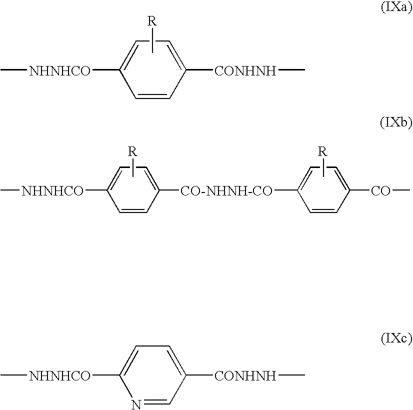
aromatic azomethines of formulae (Xa), (Xb) and (Xc) below: 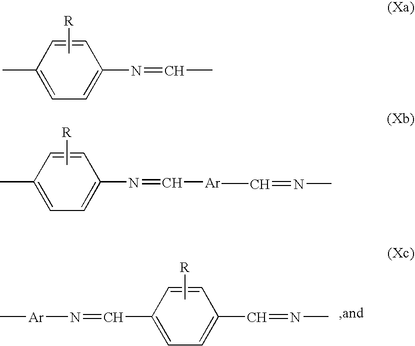
aromatic esters of formulae (XIa), (XIb) and (XIc) below: 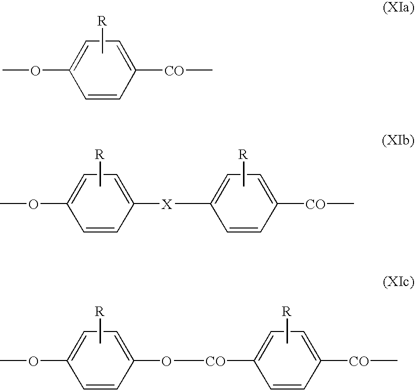
wherein, in formulae (I) to (XI):
R, R1, R2, R3, and R4, which may be identical or different, are each chosen from a hydrogen atom; —R′, —OR′, —COOR′, and —OCOR′, wherein R′ is chosen from linear and branched C₁-C₂₀ alkyl radicals; halogen atoms; nitro radicals; cyano radicals; cyanoalkyl radicals; solubilizing groups; and solubilizing groups comprising a spacer group that bonds to the ring;
Ar is chosen from radicals comprising at least one radical chosen from monoaromatic and polyaromatic radicals;
X is chosen from —NHCO—, —O—, —S—, —SO₂—, —N═N—, —C(CH₃)₂—, —CH₂—, —CH═CH— and —CH═N—; and
Z is chosen from —CH═CH— and —C≡C—.

In one embodiment, Ar is chosen from radicals comprising at least radical chosen from:

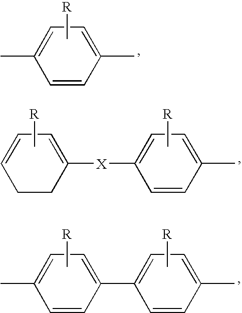

As used herein, the term “solubilizing group” means a group that ensures the dissolution of the molecule in the cosmetic medium, such that the polymer has a conductive nature after drying the composition.

It is clear that the at least one conductive polymer present in the composition disclosed herein may comprise at least one repeating unit comprising at least one solubilizing group, and at least one other repeating unit lacking the at least one solubilizing group.

The solubilizing groups may, for example, be chosen from:
carboxylic (—COOH) radicals and carboxylate (—COO-M⁺) radicals, wherein M is chosen from alkali metals, such as sodium and potassium; alkaline-earth metals, organic amines; such as a primary, secondary, and tertiary amines, alkanolamines and amino acids;
sulfonic (—SO₃H) radicals and sulfonate (—SO₃⁻M⁺) radicals, wherein M has the same definition as above;
primary, secondary, and tertiary amine radicals;
quaternary ammonium radicals, such as —N(R′)₃⁺Z⁻, wherein Z is chosen from Br, Cl, (C₁-C₄)alkyl-OSO₃ and R′, which may be identical or different, is chosen from linear and branched C₁ to C₂₀ alkyls, or two R's form a heterocycle with the nitrogen to which they are attached;
hydroxyl radicals; and
poly((C₂-C₃)alkylene oxide) radicals.

The carboxylic or sulfonic acid radicals may be optionally neutralized with a base, for example, chosen from sodium hydroxide, 2-amino-2-methylpropanol, triethylamine, and tributylamine.

The amine radicals may be optionally neutralized with a mineral acid, for example, chosen from hydrochloric acids and organic acids, such as acetic acid and lactic acid.

In addition, it should be noted that the solubilizing radicals may be connected to the ring via a spacer group, for example, chosen from —R″—, —OR″—, —OCOR″—, and —COOR″—, wherein R″ is chosen from linear and branched C₁-C₂₀ alkyl radicals optionally comprising at least one hetero atom, for example, oxygen.

In one embodiment, R, R1, R2, R3, and R4, which may be identical or different, are chosen from hydrogen, R′, —OR′, —OCOR′-COOR′, wherein R′is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

The number n of repeating units in the at least one conductive polymer may, for example, range from 5 to 10 000, further, for example, from 5 to 1000, further, for example, from 10 to 1000 and, even further, for example, from 20 to 700.

In yet another embodiment, the at least one conductive polymer is such that at least one radical chosen from R, R1, R2, R3, and R4 is a solubilizing group.

In another embodiment, the at least one conductive polymer comprises at least one solubilizing group per repeating unit. Thus, for example, the at least one radical chosen from R, R1, R2, R3, and R4 is a solubilizing group.

In another embodiment, the at least one conductive polymer is soluble in the medium of the composition.

The at least one conductive polymer present in the composition disclosed herein may, for example, be well known to those skilled in the art and may be described, for example, in the book “Handbook of Organic Conductive Molecules and Polymers”—Wiley 1997—New York, Vol 1, 2, 3, and also in the review Can. J. Chem. Vol 64, 1986.

Polythiophenes and their synthesis are, for example, described in the article taken from the review Chem. Mater. 1998, Vol. 10, No 7, pages 1990-1999 by the authors Rasmussen S. C., Pickens J. C. and Hutchison J. E. “A New, General Approach to Tuning the Properties of Functionalized Polythiophenes: The Oxidative Polymerization of Monosubstituted Bithiophenes”; in the article taken from the review Macromolecules 1998, 31, pages 933-936, by the same authors “Highly Conjugated, Water-Soluble Polymers Via Direct Oxidative Polymerization of Monosubstituted Bithiophenes”. In addition to polymerization via chemical or electrochemical oxidation, the polythiophenes may also be obtained by at least one reaction chosen from polycondensation (dihalothiophene; catalysis with nickel or palladium complexes); Suzuki coupling (coupling between a halogen functional group, for example, bromine, and a boronic acid, catalysis: palladium complex and base; this then gives coupling of AA-BB type (reaction of monomers of the type A-X-A with B-X′-B) or of A-B type (reaction of several monomers of the type A-X-B); Stille coupling (formation of a carbon-carbon bond in the presence of a Pd-based catalyst-AA-BB or A-B type); Reike polymerization (organozinc in the presence of a nickel complex); and polymerization of McCulloch type, etc.

The at least one conductive polymer present in the composition disclosed herein is moreover described in International Patent Publication No. WO 99/47570.

Examples of the at least one conductive polymer that are suitable for use in the composition disclosed herein include polymers comprising at least one repeating unit chosen from units corresponding to formulae (IIIa), (IIIb), and (IIIc) wherein the solubilizing groups may, for example, be chosen from carboxylic acid radicals; neutralized carboxylic acid radicals; sulfonic acid radicals; neutralized sulfonic acid radicals; tertiary amine radicals; and quaternary ammonium radicals, such as —N(R′)₃⁺Z⁻, wherein Z is chosen from Br, Cl, (C₁-C₄)alkyl-OSO₃ and R′, which may be identical or different, is chosen from linear and branched C₁ to C₂₀ alkyls, or two R's form a heterocycle with the nitrogen to which they are attached; wherein the groups are optionally connected to the ring via a spacer group.

Thus, the polymerization may be performed via chemical or electrochemical oxidation of the corresponding thiophene monomer or via polycondensation.

For example, the polythiophenes of formulae (IIIa) and (IIIb) may be obtained by at least one reaction chosen from the following:
polymerization via oxidation (for example with FeCl₃ catalysis);
polycondensation of dihalothiophene catalysed with nickel or palladium complexes (e.g.: NiCl₂(dppe)₂);
Suzuki coupling (coupling between a halogen functional group, for example, bromine, and a boronic acid, catalysis: palladium complex and base; this then gives coupling of AA-BB type (reaction of monomers of the type A-X-A with B-X′-B) or of A-B type (reaction of several monomers of the type A-X-B);
Stille coupling (formation of a carbon-carbon bond formed in the presence of a Pd-based catalyst—AA-BB or A-B type);
Reike polymerization (organozinc in the presence of a nickel complex); and
polymerization of McCulloch type, etc.

The vinylene polythiophenes of formula (IIIc) wherein Z is —CH═CH— may be obtained by at least one reaction chosen, for example, from Gilch polymerization in the presence of a strong base (potassium tert-butoxide) of 2,5-bis(bromoalkylene)thiophene; polymerization by the Wessling method via the use of a precursor based on sulfonium salts and pyrolysis; and a Wittig-Horner Wittig reaction.

The ethynylene polythiophenes of formula (IIIc) wherein Z is —C≡C— may be obtained by at least one reaction chosen from Heck-Sonogashira coupling (of the type AA-BB or A-B; formation of a carbon-carbon bond between a terminal acetylenic (or true acetylenic) functional group and a bromo or iodo functional group, catalysed with a palladium/copper complex (PdCl₂(PPh₃)₃, CuI or Cu(OAc)₂) in the presence of a base such as triethylamine, diisopropyl amine, piperidine, etc.); and metathesis of alkynes in the presence of a molybdenum complex (Mo(CO)₆).

In general, the functionalization of the polythiophenes, in other words the introduction of the solubilizing or non-solubilizing group(s), is performed on the monomer before it is polymerized.

In some embodiments, the solubilizing group may be obtained after working up the polymer. This may, for example, be the case for the carboxylic acid functional group, which may be obtained by hydrolysis of the corresponding ester.

For example, in one embodiment, the solubilizing groups are chosen from carboxylic acid radicals; neutralized carboxylic acid radicals; sulfonic acid radicals; neutralized sulfonic acid radicals; tertiary amine radicals; quaternary ammonium radicals, such as —N(R′)₃+Z wherein Z is chosen from Br, Cl, (C₁-C₄)alkyl-OSO₃ and R′, which may be identical or different, is chosen from linear and branched C₁-C₂₀ alkyl radicals, optionally connected to the ring via a spacer group, for example, chosen from C₁-C₂₀ alkyl radicals; and salts thereof.

In another embodiment, the at least one conductive polymer comprises at least one repeating unit chosen from units of formulae (IIIa), (IIIb) and (IIIc), wherein at least one radical chosen from R1, R2, R3, and R4 of formula (IIIa) and R1 and R2 of formulae (IIIb) and (IIIc) is chosen from carboxylic acid solubilizing groups, in neutralized or non-neutralized form, optionally connected to the ring via a spacer group, for example, chosen from linear and branched C₁-C₂₀ alkyl radicals, wherein the other radical(s) is (are) hydrogen(s).

The at least one conductive polymer may be present in the composition in an amount greater than or equal to 0.001% by weight, for example, greater than or equal to 0.01% by weight, further, for example, greater than or equal to 0.1% by weight and, even further, for example, greater than or equal to 0.5% by weight, relative to the total weight of the composition. Moreover, the at least one conductive polymer may be present in an amount less than or equal to 50% by weight, for example, less than or equal to 30% by weight, further, for example, less than or equal to 20% by weight and, even further, for example, less than or equal to 10% by weight, relative to the total weight of the composition.

In another embodiment, the at least one conductive polymer is present in an amount ranging from 0.1% to 50% by weight, for example, from 0.1% to 30% by weight and, further, for example, from 0.5% to 10% by weight, relative to the total weight of the composition.

Oxidizing Agent

As indicated above, the composition disclosed herein comprises at least one oxidizing agent in addition to the at least one conductive polymer.

In one embodiment, the at least one oxidizing agent is chosen from peroxygenated compounds, such as ammonium and alkali metal persulfates; perborates and percarbonates; urea peroxide; halogenated derivates, such as iodine, alkali metal bromates, and alkali metal hypochlorites, for example, alkali metal ferricyanides; and enzymes, such as peroxidases and two-electron and four-electron oxidoreductases.

In another embodiment, the composition comprises hydrogen peroxide as the at least one oxidizing agent.

In yet another embodiment, the composition comprises peroxygenated compounds, such as ammonium and alkali metal persulfates; perborates and percarbonates; urea peroxide; halogenated derivates, such as iodine, alkali metal bromates, and alkali metal hypochlorites, for example, alkali metal ferricyanides; enzymes, such as peroxidases and two-electron and four-electron oxidoreductases; and hydrogen peroxide.

The at least one oxidizing agent may be present in an amount ranging from 0.05% to 25% by weight, relative to the total weight of the composition.

In yet another embodiment, the composition may comprise at least one alkaline agent, in cases where the composition disclosed herein does not comprise hydrogen peroxide, or does not comprise both water and a peroxygenated salt.

The at least one alkaline agent may be chosen from urea; alkali metal and alkaline-earth metal silicates and phosphates; ammonia-precursor compounds; alkaline carbonates; alkanolamines, such as monoethanolamine, diethanolamine and triethanolamine, and derivatives thereof; sodium hydroxide; potassium hydroxide; and compounds of formula (A) below: 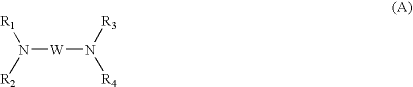
wherein:
W is a propylene residue optionally substituted with at least one entity chosen from hydroxyl groups and C₁-C₆ alkyl radicals and R1, R2, R3 and R4, which may be identical or different, are each chosen from a hydrogen atom, C₁-C₆ alkyls, and C₁-C₆ hydroxyalkyls.

The at least one alkaline agent may be present in an amount ranging from 0.01% to 40% by weight and, for example, from 0.1% to 30% by weight, relative to the total weight of the composition.

The composition may also comprise at least one surfactant chosen from nonionic, anionic, cationic, amphoteric, and zwitterionic surfactants. Examples of the at least one surfactant include alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and hydroxypropyl ether nonionic surfactants.

If present, the least one surfactant may be present in an amount less than or equal to 30% by weight and, for example, ranging from 0.5% to 10% by weight, relative to the weight of the composition.

The pH of the composition may range, for example, from 1.5 to 12 and, further, for example, from 2.5 to 11. It may be adjusted to the desired value using acidifying or basifying agents.

The basifying agents have been described above for the at least one alkaline agent.

Examples of acidifying agents include mineral and organic acids, such as hydrochloric acid, orthophosphoric acid, and sulfuric acid; sulfonic acids; and carboxylic acids, for example, acetic acid, tartaric acid, citric acid, and lactic acid.

The composition may also comprise at least one conventionally used adjuvant, for example, chosen from thickeners and gelling agents, such as hydrophilic and hydrophobic fumed silica; nonionic, anionic, and amphoteric associative polymers; and water-soluble thickening polymers of synthetic and natural origin, such as polyvinylpyrrolidone, polyacrylic acid, polyacrylamide and polysaccharides of animal, plant, and microbial origin.

In one embodiment, the thickeners may be present in an amount ranging from 0.01% to 10% by weight and, for example, from 0.05% to 5% by weight, relative to the total weight of the composition.

The composition may also comprise at least one conventionally used adjuvant chosen from conditioners, such as cationic and amphoteric conditioners, which may, for example, be anhydrous; fillers, such as clay and amorphous silica; binders, such as vinylpyrrolidone; lubricants, such as polyol stearates, alkali metal and alkaline-earth metal stearates; agents for controlling the release of oxygen, such as magnesium carbonate and magnesium oxide; dyes; matting agents, such as titanium oxides; antioxidants; penetrating agents; sequestering agents; fragrances; buffers, dispersants; film-forming agents, ceramides; preserving agents; and stabilizers, etc.

In one embodiment, the at least one conventionally used adjuvant is present in an amount less than or equal to 60% by weight, relative to the total weight of the composition.

The composition disclosed herein may be provided in various forms.

In one embodiment, the composition may be in the form of a composition free of water or a composition comprising a low water content. For example, a composition that comprises less than 1% by weight of water and, for example, less than 0.5% by weight of water, relative to the total weight of the composition is considered low.

In another embodiment, the composition may be in the form of a paste or granules.

In yet another embodiment, the composition comprises a cosmetically acceptable medium, for example, chosen from water and mixtures of water and at least one cosmetically acceptable organic solvent. The at least one cosmetically acceptable organic solvent may be chosen from C₁-C₄ alcohols, such as ethyl alcohol and isopropyl alcohol; aromatic alcohols, such as benzyl alcohol and phenylethyl alcohol; glycols and glycol ethers, such as ethylene glycol monomethyl, monoethyl and monobutyl ether, propylene glycol and ethers thereof such as, propylene glycol monomethyl ether, butylene glycol, dipropylene glycol, diethylene glycol alkyl ethers, such as diethylene glycol monoethyl ether and monobutyl ether; and polyols, such as glycerol. Polyethylene glycols and polypropylene glycols and mixtures of all these compounds may also be used as the at least one cosmetically acceptable organic solvent. If present, the at least one cosmetically acceptable organic solvent may be present in an amount ranging from 1% to 40% by weight and, for example, from 5% to 30% by weight, relative to the total weight of the composition.

Also disclosed herein is ready-to-use composition comprising in a cosmetically acceptable medium,
(a) at least one oxidizing agent and
(b) at least one conductive polymer, wherein the at least one oxidizing agent is chosen from peroxygenated compounds, alkali metal bromates, and hydrogen peroxide.

The processes using the composition disclosed herein will now be described. The processes are applicable to keratin fibers, for example, human keratin fibers, such as hair.

Thus, one embodiment relates to a process comprising applying the composition that has been described to the keratin fibers.

Another embodiment relates to a process for bleaching keratin fibers comprising:
(a) mixing, before application to the keratin fibers,
   at least one composition comprising in a cosmetically acceptable medium:
      (i) at least one oxidizing agent and
      (ii) at least one conductive polymer, wherein the at least one composition is free of hydrogen peroxide and
   (ii) at least one aqueous composition comprising hydrogen peroxide, wherein the compositions are optionally mixed in the presence of at least one alkaline agent;
b) applying the mixture to wet or dry keratin fibers;
c) leaving the mixture on the fibers for a time period that is sufficient for the desired bleaching effect to take place;
d) optionally rinsing the fibers;
e) optionally washing the fibers and rinsing the fibers; and
f) drying the fibers or leaving the fibers to dry.

During step a), the at least one composition may be mixed with at least one aqueous composition, before applying the mixture to the fibers.

In one embodiment, the at least one composition comprises, as the at least one oxidizing agent, peroxygenated compound as defined above.

In one embodiment, the at least one composition may be mixed with 0.5 to 10 weight equivalents of the at least one aqueous composition.

The at least one aqueous composition may be in the form of a solution, an emulsion, or a gel.

In addition, the hydrogen peroxide may be present in an amount ranging from 2% to 12% by weight, relative to the total weight of the at least one aqueous composition.

It should be noted that the at least one aqueous composition may, for example, have a pH of less than 7 so as to ensure the stability of this composition.

This pH value is conventionally obtained using acidifying or basifying agents as described previously.

The at least one aqueous composition may also comprise at least one additive chosen from preserving agents, dyes, fragrances, antifoams, hydrogen peroxide stabilizers, such as sodium pyrophosphate, sodium stannate and sodium salicylate, and sequestering agents.

As mentioned above, the mixture prepared during step a) optionally comprises at least one alkaline agent. In one embodiment, the at least one alkaline agent may be present in the at least one composition or may be added during the mixing performed in step a).

In one embodiment, the amount of the at least one alkaline agent is such that the pH of the mixture obtained after step a) ranges from 7 to 11.5 and, for example, from 8 to 11.

The leave-in time period of the mixture during step c) may range from 3 and 30 minutes and, for example, from 5 to 20 minutes.

Moreover, and purely as a guide, step c) may be performed at a temperature ranging from 15 to 80° C. and, for example, from 20 to 40° C.

Step f) of the process may, for example, be performed at a temperature ranging from 20 to 100° C. and, for example, from 20 to 80° C.

Another embodiment disclosed herein relates to a process for permanent-reshaping of keratin fibers comprising,
a) applying to the fibers at least one reducing composition comprising at least one reducing agent;
b) leaving the at least one reducing composition on the fibers for a time period that is sufficient to shape the fibers;
c) optionally rinsing the fibers;
d) applying to the fibers at least one composition comprising, in a cosmetically acceptable medium,
   (i) at least one oxidizing agent and
   (ii) at least one conductive polymer;
b) optionally rinsing the fibers;
c) optionally washing the fibers and rinsing the fibers; and
d) drying the fibers or leaving the fibers to dry.

In one embodiment, step d) is performed on fibers that have been treated beforehand with at least one reducing composition comprising at least one reducing agent. The at least one reducing agent may be chosen from reductones and sulfur-comprising reducing agents, for example, compounds comprising at least one functional group chosen from thiol, (di)sulfide, (di)sulfite, and hydrosulfite functional groups.

Examples of suitable sulfur-comprising compounds include thioglycolic acid, thiolactic acid, alkali metal and alkaline-earth metal salts thereof (such as sodium, potassium, and calcium salts) and esters thereof; β-mercaptoethanol; cysteine and cysteamine, and derivatives thereof; homocysteine and a salt thereof; mercaptoaldehyde; penecillamine; glutathione, cystine; and alkali metal, alkaline-earth metal, ammonium sulfite, bisulfite, hydrosulfite, and metabisulfite. Examples of reductones include ascorbic acid, isoascorbic acid, and salts and esters thereof; hydroxypropanedial, and 2,3-hydroxy-2-cyclopenten-1-one.

The at least one reducing agent may be present in an amount ranging from 1% to 30% by weight and, for example, from 5% to 20% by weight, relative to the total weight of the at least one reducing composition.

The at least one reducing composition may comprise water or mixtures of water and at least one cosmetically acceptable solvent chosen, for example, from those solvents described for the composition disclosed herein comprising water.

During the reduction step, the keratin fibers may be shaped, in other words smoothed out or placed under tension using curlers, before or after the application of the at least one reducing composition, or simultaneously therewith.

The leave-in time period may range, for example, from 3 to 30 minutes and, for example, from 5 to 20 minutes. Moreover, and purely as a guide, step d) may be performed at a temperature ranging from 15 to 60° C. and, for example, from 20 to 40° C.

Once the reduction step has been performed, the fibers may, for example, be rinsed with water and the at least one composition disclosed herein may be applied. The at least one composition used during this step may, for example, be an aqueous composition. Thus, in one embodiment, in the case where the at least one composition used is in a form that is free of water or that has a low water content, the at least one composition is mixed with an aqueous solution before application to the fibers. This aqueous solution may or may not comprise at least one alkaline agent such as those described above.

The leave-in time period for the shape-fixing step b) may, for example, range from 3 to 30 minutes and, for example, from 5 to 20 minutes. Moreover, and purely as a guide, step b) may be performed at a temperature ranging from 15 to 80° C. and, for example, from 20 to 40° C.

Another embodiment disclosed herein relates to a process for dyeing keratin fibers comprising,
a) mixing, before application to the keratin fibers,
   at least one dye composition comprising at least one dye chosen from oxidation dye precursors and direct dyes and
   at least one aqueous composition comprising, in a cosmetically acceptable medium,
      (i) at least one oxidizing agent and
      (ii) at least one conductive polymer, wherein the compositions are mixed in the presence of at least one alkaline agent;
b) applying the mixture to the wet or dry keratin fibers;
c) leaving the mixture on the fibers for a time period that is sufficient for the desired dyeing effect to take place;
d) optionally rinsing the fibers;
e) optionally washing the fibers and rinsing the fibers; and
f) drying the fibers or leaving the fibers to dry.

During step a) the at least one composition disclosed herein may, for example, comprise hydrogen peroxide as the at least one oxidizing agent, with at least one conductive polymer as described above.

The at least one composition is mixed with the at least one dye composition comprising at least one dye chosen from oxidation dye precursors and direct dyes.

The oxidation dye precursors forming part of the at least one dye composition intended to be mixed with the at least one composition may be chosen from compounds that are conventionally used in the field. For example, the at least one dye composition may comprise at least one oxidation base optionally combined with at least one coupler.

Examples of the at least one oxidation base include ortho- and para-phenylenediamines, double bases, ortho- and para-aminophenols, heterocyclic bases, and addition salts thereof with an acid. If present, the at least one oxidation base may, for example, be present in an amount ranging from 0.0005% to 12% by weight and, for example, from 0.005% to 8% by weight, relative to the total weight of the at least one dye composition.

Examples of the at least one coupler include meta-aminophenols, meta-phenylenediamines, meta-diphenols, naphthols, heterocyclic couplers, and addition salts thereof with an acid. If present, the at least one coupler may, for example, be present in an amount ranging from 0.0001% to 10% by weight and, for example, from 0.005% to 5% by weight, relative to the total weight of the at least one dye composition.

The direct dyes may be chosen from nooninic, cationic, and anionic direct dyes. Examples of the direct dyes include nitrobenzene dyes, azo, azomethine, methine, anthraquinone, naphthoquinone, benzoquinone, phenothiazine, indigoid, xanthene, phenanthridine, phthalocyanin and triarylmethane-based dyes, and natural dyes, alone or as mixtures.

Furthermore, if the at least one dye composition comprises direct dyes, they may be present in an amount ranging from 0.0005% to 12% by weight and, for example, from 0.005% to 6% by weight, relative to the total weight of the at least one dye composition.

The at least one dye composition comprises a cosmetically acceptable medium, for example, chosen from water and mixtures of water and at least one solvent. The at least one dye composition may also comprise at least one additive chosen from those which are previously known elsewhere in the treatment of keratin fibers. For example, the at least one additive may be chosen from surfactants that are well known in the state of the art, such as anionic, cationic, nonionic, amphoteric, and zwitterionic surfactants; thickeners; antioxidants; fragrances; dispersants; conditioners; such as cationic and amphoteric polymers; opacifiers; sequestering agents, such as EDTA and etidronic acid; UV-screening agents; waxes; volatile and non-volatile, linear, branched and cyclic silicones, which may be organomodified (for example, with at least one amine group) and unmodified; preserving agents; ceramides; pseudoceramides; plant, mineral, and synthetic oils; vitamins and provitamins, such as panthenol; and nonionic, anionic, amphoteric, and cationic associative polymers.

As mentioned previously, in one embodiment, the mixing of the at least one dye composition and of the at least one composition disclosed herein takes place in the presence of at least one alkaline agent.

That which has been stated in the context of the other embodiments of the processes disclosed herein, and also the pH conditions, remain valid and reference may be made thereto.

However, in one embodiment, the at least one alkaline agent may be present in the at least one dye composition.

The temperature at which the at least one composition is applied during step b) may, for example, range from 15 to 80° C. and, for example, from 15 to 40° C. The leave-in time period may range, for example, from 5 to 60 minutes and, for example, from 5 to 40 minutes. Once this step has been performed, the fibers may be optionally rinsed, then, for example, washed with a shampoo, and then rinsed again. Finally, the fibers may be dried or left to dry.

Other than in the example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed composition. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific example is reported as precisely as possible. Any numerical value, however, inherently contain certain errors necessarily resulting from the standard deviation found in their respective testing measurements. The following example is intended to illustrate the present disclosure without limiting the scope as a result.

### EXAMPLE

### Synthesis of Poly(thiophene-3-acetic acid)

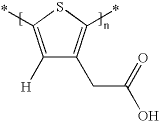

Procedure

Preparation of the polymer: poly(ethyl thiophene-3-acetate)

25 ml of dry chloroform was introduced into a Schienk tube under argon, the system was degassed and the following reagents were then introduced:
2.5 g of ethyl thiophene-3-acetate (14.7 mmol) 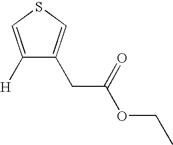
   and 1 g of FeCl₃ (6.15 mmol).

The mixture was stirred for 24 hours under argon at 50° C.

The poly(ethyl thiophene-3-acetate) polymer was then precipitated in heptane. The polymer was then dissolved in a tetrahydrofuran solution.

Infrared Characterization:

C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the polymer: poly(ethyl thiophene-3-acetate) to form poly(thiophene-3-acetic acid)

The polymer obtained above was then hydrolysed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was then filtered off and washed several times with distilled water in order to remove the traces of catalyst.

Infrared Characterization of the Polymer:
C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation Comprising the Polymer and Process for Using the Formulation:

| | |
|---|---|
| Poly(thiophene-3-acetic acid) | 5 g |
| Hydrogen peroxide qs | 20 volumes |
| 20% aqueous ammonia qs | 9 g |
| Ethyl alcohol | 20 g |
| Water qs | 100 g |

The formula was applied to dark hair. After a standing time of 20 minutes, drying (drying in air) was performed.

What is claimed is:

1. A composition comprising, in a cosmetically acceptable medium,
(a) at least one oxidizing agent and
(b) at least one conductive polymer.

2. The composition according to claim 1, wherein the at least one conductive polymer comprises at least one repeating unit chosen from:
anilines of formula (I) below: 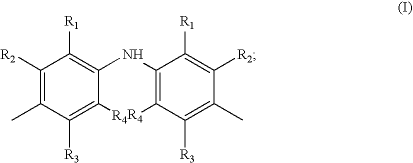
pyrroles of formulae (IIa) and (IIb) below: 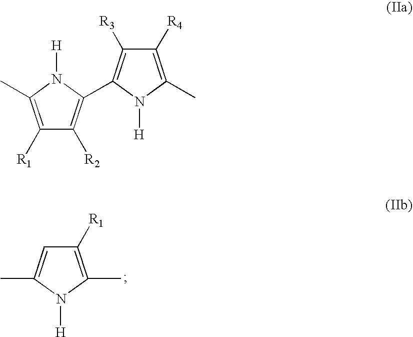
thiophenes and bisthiophenes of formulae (IIIa), (IIIb) and (IIIc) below: 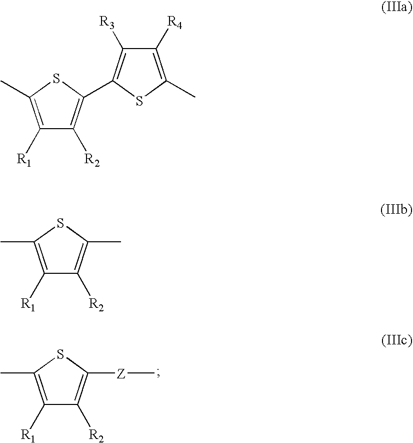
furans of formula (IV) below: 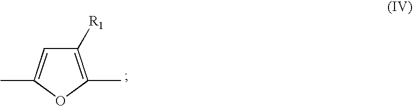
para-phenylene sulfides of formula (V) below: 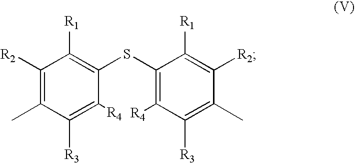
para-phenylenevinylenes of formula (VI) below: 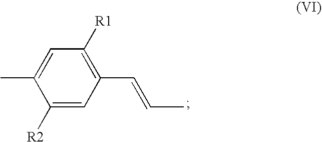
indoles of formula (VII) below: 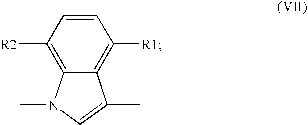
aromatic amides of formulae (VIIIa), (VIIIb), (VIIIc) and (VIIId) below: 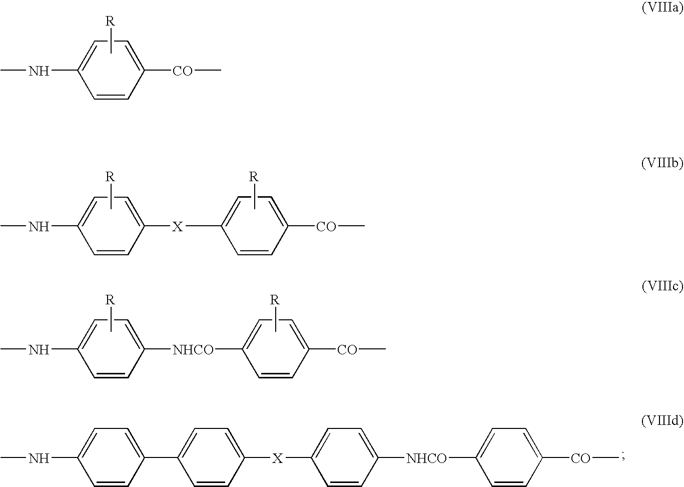
aromatic hydrazides of formulae (IXa), (IXb) and (IXc) below: 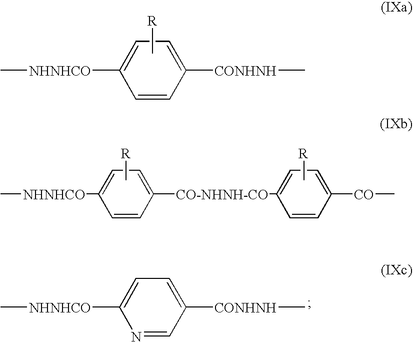
aromatic azomethines of formulae (Xa), (Xb) and (Xc) below: 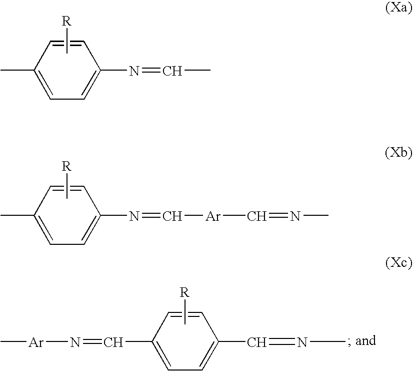
aromatic esters of formulae (XIa), (XIb) and (XIc) below: 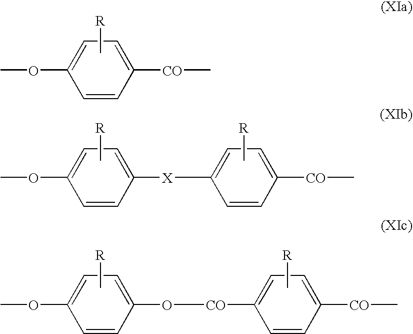
wherein, in formulae (I) to (XI):
R, R1, R2, R3, and R4, which may be identical or different, are each chosen from a hydrogen atom, —R′, —OR′, —COOR′, —OCOR′, wherein R′ is chosen from linear and branched C₁-C₂₀ alkyl radicals, halogen atoms, nitro radicals, cyano radicals, cyanoalkyl radicals, solubilizing groups, and solubilizing groups comprising a spacer group that bonds to the ring;
Ar is chosen from radicals comprising at least one radical chosen from monoaromatic and polyaromatic radicals;
X is chosen from —NHCO—, —O—, —S—, —SO₂—, —N═N—, —C(CH₃)₂—, —CH₂—, —CH═CH— and —CH═N—; and
Z is chosen from —CH═CH— and —C≡C—.

3. The composition according to claim 2, wherein the solubilizing groups are chosen from:
carboxylic (—COOH) and carboxylate (—COO-M⁺), wherein M is chosen from alkali metals, alkaline-earth metals, organic amines, alkanolamines, and amino acids;
sulfonic (—SO₃H) and sulfonate (—SO₃⁻M⁺), wherein M is chosen from alkali metals, alkaline-earth metals, organic amines, alkanolamines, and amino acids;
primary, secondary, and tertiary amines;
quaternary ammonium radicals;
hydroxyls; and
poly((C₂-C₃)alkylene oxides.

4. The composition according to claim 3, wherein the quaternary ammonium radicals are chosen from —N(R′)₃⁺Z⁻ groups, wherein Z is chosen from Br, Cl, and (C₁-C₄)alkyl-OSO₃ and R′, which may be identical or different, is chosen from linear and branched C₁ to C₂₀ alkyls, or two of the R's form a heterocycle with the nitrogen to which they are attached.

5. The composition according to claim 2, wherein the solubilizing groups are connected to the ring via a spacer group.

6. The composition according to claim 5, wherein the spacer group is chosen from —R″—, —OR″—, —OCOR″—, and —COOR″—, wherein R″ is chosen from linear and branched C₁-C₂₀ alkyl radicals optionally comprising at least one hetero atom.

7. The composition according to claim 2, wherein the radicals R, R1, R2, R3, and R4, which may be identical or different, are each chosen from hydrogen, R′, —OR′, —OCOR′—, and COOR′, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]CH₂CH₂OCH₃, wherein x is an average number ranging from 0 to 200.

8. The composition according to claim 2, wherein at least one radical chosen from R, R1, R2, R3, and R4 of the at least one conductive polymer is a solubilizing group.

9. The composition according to claim 2, wherein the at least one conductive polymer comprises at least one solubilizing group per repeating unit.

10. The composition according to claim 2, wherein the solubilizing groups are chosen from carboxylic acid radicals; neutralized carboxylic acid radicals; sulfonic acid radicals; neutralized sulfonic acid radicals; tertiary amine radicals; quaternary ammonium radicals, wherein the solubilizing groups are optionally connected to the ring via a spacer group; and salts thereof.

11. The composition according to claim 10, the quaternary ammonium radicals are chosen from —N(R′)₃⁺Z⁻ groups, wherein Z is chosen from Br, Cl, (C₁-C₄)alkyl-OSO₃ and R′, which may be identical or different, is chosen from linear and branched C₁-C₂₀ alkyl radicals.

12. The composition according to claim 11, wherein the spacer group is chosen from C₁-C₂₀ alkyl radicals.

13. The composition according to claim 2, wherein the at least one conductive polymer comprises at least one repeating unit chosen from units of formulae (IIIa), (IIIb) and (IIIc), wherein at least one radical chosen from R1, R2, R3, and R4 of formula (IIIa) and R1 and R2 of formulae (IIIb) and (IIIc) is chosen from carboxylic acid solubilizing groups, in neutralized or non-neutralized form, optionally connected to the ring via a spacer group, and wherein the other radical(s) is (are) hydrogen(s).

14. The composition according to claim 13, wherein the spacer group is chosen from linear and branched C₁-C₂₀ alkyl radicals.

15. The composition according to claim 1, wherein the at least one conductive polymer is present in an amount greater than or equal to 0.001% by weight, relative to the total weight of the composition.

16. The composition according to claim 15, wherein the at least one conductive polymer is present in an amount less than or equal to 50% by weight, relative to the total weight of the composition.

17. The composition according to claim 15, wherein the at least one conductive polymer is present in an amount ranging from 0.1% to 50% by weight, relative to the total weight of the composition.

18. The composition according to claim 1, wherein the at least one conductive polymer has a conductivity ranging from 1×10⁻⁵ to 5×10⁵ siemens/cm.

19. The composition according to claim 18, wherein the at least one conductive polymer has a conductivity ranging from 1×10⁻³ to 1×10⁵ siemens/cm.

20. The composition according to claim 19, wherein the at least one conductive polymer has a conductivity ranging from 1×10⁻¹ to 1×10⁴ siemens/cm.

21. The composition according to claim 1, wherein the at least one oxidizing agent is chosen from peroxygenated compounds; halogenated derivatives; alkali metal ferricyanides; and enzymes.

22. The composition according to claim 1, wherein the at least one oxidizing agent is hydrogen peroxide.

23. The composition according to claim 1, wherein the at least one oxidizing agent is present in an amount ranging from 0.05% to 25% by weight, relative to the total weight of the composition.

24. The composition according to claim 1, further comprising at least one alkaline agent.

25. The composition according to claim 24, wherein the at least one alkaline agent is chosen from urea, alkali metal and alkaline-earth metal silicates and phosphates, and ammonia-precursor compounds.

26. The composition according to claim 1, wherein the composition does not comprise water.

27. The composition according to claim 1, wherein the composition comprises less than 1% by weight of water, relative to the total weight of the composition.

28. The composition according to claim 1, wherein the cosmetically acceptable medium is chosen from water and mixtures of water and at least one cosmetically acceptable organic solvent.

29. The composition according to claim 28, wherein the at least one cosmetically acceptable organic solvent is chosen from C₁-C₄ alcohols, aromatic alcohols, glycols, glycol ethers, polyols, polyethylene glycols, and polypropylene glycol.

30. The composition according to claim 1, further comprising at least one surfactant chosen from nonionic, anionic, cationic, amphoteric, and zwitterionic surfactants.

31. The composition according to claim 30, wherein the at least one surfactant is present in an amount less than or equal to 30% by weight, relative to the total weight of the composition.

32. The composition according to claim 31, wherein the at least one surfactant is present in an amount ranging from 0.5% to 10% by weight, relative to the weight of the composition.

33. A ready-to-use composition comprising,
a composition comprising, in a cosmetically acceptable medium,
(a) at least one oxidizing agent and
(b) at least one conductive polymer; wherein the at least one oxidizing agent is chosen from peroxygenated compounds, alkali metal bromates, and hydrogen peroxide.

34. A process for treating keratin fibers comprising applying at least one composition comprising, in a cosmetically acceptable medium,
(a) at least one oxidizing agent and
(b) at least one conductive polymer.

35. A process for bleaching keratin fibers comprising,
(a) mixing, before application to the fibers,
at least one composition comprising in a cosmetically acceptable medium:
(i) at least one oxidizing agent and
(ii) at least one conductive polymer, wherein the at least one composition is free of hydrogen peroxide and
(ii) at least one aqueous composition comprising hydrogen peroxide, wherein the compositions are optionally mixed in the presence of at least one alkaline agent;
b) applying the mixture to wet or dry keratin fibers;
c) leaving the mixture on the fibers for a time period that is sufficient for the desired bleaching effect to take place;
d) optionally rinsing the fibers;
e) optionally washing the fibers and rinsing the fibers; and
f) drying the fibers or leaving the fibers to dry.

36. The process according to claim 35, wherein the keratin fibers are human keratin fibers.

37. The process according to claim 36, wherein the human keratin fibers are hair.

38. A process for permanently reshaping keratin fibers comprising,
a) applying to the fibers at least one reducing composition comprising at least one reducing agent;
b) leaving the at least one reducing composition on the fibers for a time period that is sufficient to shape the fibers;
c) optionally rinsing the fibers;
d) applying to the fibers at least one composition comprising, in a cosmetically acceptable medium,
(i) at least one oxidizing agent and
(ii) at least one conductive polymer;
b) optionally rinsing the fibers;
c) optionally washing the fibers and rinsing the fibers; and
d) drying the fibers or leaving the fibers to dry.

39. The process according to claim 38, wherein the keratin fibers are human keratin fibers.

40. The process according to claim 39, wherein the human keratin fibers are hair.

41. A process for dyeing keratin fibers comprising,
a) mixing, before application to the keratin fibers,
at least one dye composition comprising at least one dye chosen from oxidation dye precursors and direct dyes and
at least one aqueous composition comprising, in a cosmetically acceptable medium,
(i) at least one oxidizing agent and
(ii) at least one conductive polymer, wherein the compositions are mixed in the presence of at least one alkaline agent;
b) applying the mixture to the wet or dry keratin fibers;
c) leaving the mixture on the fibers for a time period that is sufficient for the desired dyeing effect to take place;
d) optionally rinsing the fibers;
e) optionally washing the fibers and rinsing the fibers; and
f) drying the fibers or leaving the fibers to dry.

42. The process according to claim 41, wherein the keratin fibers are human keratin fibers.

43. The process according to claim 42, wherein the human keratin fibers are hair.

44. A method for imparting at least one optical effect on keratin fibers comprising applying to the fibers, at least one composition comprising, in a cosmetically acceptable medium, at least one conductive polymer and at least one oxidizing agent.

45. The method according to claim 44, wherein the at least one optical effect is sheen.

46. A multi-compartment kit comprising,
at least one compartment comprising at least one composition comprising, in a cosmetically acceptable medium, at least one conductive polymer and at least one oxidizing agent and
at least one second compartment comprising at least one composition comprising, dyes, reducing agents, or hydrogen peroxide.
